(19) United States
(12) Patent Application Publication
(10) Pub. No.: US 2013/0316028 A1
(43) Pub. Date: Nov. 28, 2013

(54) FREEZE DRIED EXTRACT OF PARKIA, PREFERABLY OF PARKIA SPECIOSA BEANS FOR TREATMENT OF DISEASES SUCH AS DIABETES MELLITUS TYPE 2

(71) Applicants: Alberta Berendina Buring, Hellendoorn (NL); Albertus Justus Johannes Van Den Berg, Utrecht (NL)

(72) Inventors: Alberta Berendina Buring, Hellendoorn (NL); Albertus Justus Johannes Van Den Berg, Utrecht (NL)

(21) Appl. No.: 13/985,797

(22) PCT Filed: Feb. 15, 2012
(86) PCT No.: PCT/EP2012/000662

§ 371 Date: Aug. 15, 2013

(30) Foreign Application Priority Data
Feb. 15, 2011 (EP) 11001210.1

(51) Int. Cl. A61K 36/48

(52) U.S. Cl. CPC A61K 36/48
USPC 424/757

(57) ABSTRACT

The invention relates to a process for the preparation of a freeze-dried fruit of Parkia, preferably Parkia speciosa comprising the steps of a) freezing a fruit, preferably beans from Parkia to −20° C. or below to obtain a frozen fruit and b) subsequently freeze-drying the frozen fruit by reducing the pressure surrounding the said frozen fruit to allow water and volatiles to sublimate and/or evaporate from the frozen fruit to obtain the freeze-dried fruit of Parkia, wherein the temperature of the fruit during freeze-drying is at most 0° C., wherein the temperature of the fruit from the beginning of step a) until the end of step b) does not exceed 30° C. The invention also relates to the products obtained with the process, including their use as a medicament in the treatment, co-treatment and/or prevention of diabetes mellitus and/or for weight regulation.

The invention relates to a process for the preparation of a freeze-dried fruit of Parkia, the product obtained with said process, a pharmaceutical composition comprising said product, a nutraceutical composition comprising said product, said pharmaceutical composition for use as a medicament, preferably for use in the treatment, co-treatment and/or prevention of a disease chosen from the group of diabetes mellitus type 2, diseases related to β-amyloid plaque formation and neurodegenerative diseases, preferably of diabetes mellitus type 2, to a method of treatment, co-treatment or prevention of these diseases in animals using said product or said composition and to use of said product or said composition for regulation of weight in animals, preferably humans.

Diabetes mellitus, commonly called diabetes, refers to a disease process derived from multiple causative factors and is characterized by elevated levels of plasma glucose, a condition referred to as hyperglycaemia. According to the American Diabetes Association, diabetes mellitus is estimated to affect approximately 6% of the world population. Uncontrolled hyperglycaemia is associated with increased and premature mortality due to an increased risk for microvascular and macrovascular diseases, including nephropathy, neuropathy, retinopathy, hypertension, cerebrovascular disease and coronary heart disease. Therefore, control of hyperglycaemia is an important aspect in treating diabetes.

There are two major forms of diabetes: Type 1 diabetes and Type 2 diabetes. Type 1 diabetes is the result of an absolute deficiency of insulin, the hormone which regulates glucose utilization. Such absolute insulin deficiency is usually characterized by β-cell destruction within the Islets of Langerhans in the pancreas. Type 2 diabetes is a disease characterized by insulin resistance accompanied by relative, rather than absolute, insulin delivery. Type 2 diabetes can range from predominant insulin resistance with relative insulin deficiency to predominant insulin deficiency with some insulin resistance. Insulin resistance is the diminished ability of insulin to exert its biological action across a broad range of concentrations. In insulin resistant individuals, the body secreted abnormally high amounts of insulin to compensate for this effect. When inadequate amounts of insulin are present to compensate for insulin resistance leading to inadequeate control of glucose, a state of impaired glucose tolerance develops. In a significant number of individuals, insulin secretion then declines and the plasma glucose level rises even further, resulting in a clinical state of diabetes.

The incidence of type 2 diabetes is increasing worldwide. Although genetic factors may play a role, life-style changes such as increased fat intake and reduced exercise are associated with obesity and insulin resistance. Biguanides, such as metformin, are commonly used, either alone, or in combination with insulin, for treatment of type 2 diabetes.

However, the medication that is conventionally used for the treatment of type 2 diabetes, such as metformin and optionally also insulin is not available to the entire world population, mainly due to its costs. Furthermore, the medications currently in use have adverse side-effects.

Therefore, and especially for the third world population, there is a desire to search for more affordable and widely available or easy to produce medication for treatment of type 2 diabetes. Scientists are therefore investigating the efficacy of indigenous plans in their own country.

An example of a fruit of a plant that is known to be effective in the treatment of diabetes mellitus is the Parkia speciosa, also known as petai, bitter bean, sataw, twisted cluster bean, yongchaa, yongchaak or kampai, zawngtah or stink bean. The Parkia speciosa is a plant of the genus Parkia in the family Fabaceae. It bears long, flat edible pods containing bright green beans the size and shape of plump almonds.

A disadvantage of the beans from Parkia speciosa is that they have a rather peculiar smell, which is the reason why the Parkia speciosa has earned its nickname ‘stink bean’ because its strong smell is very pervasive and lingers in the mouth and the excretory systems of the body. After consumption of the Parkia speciosa beans one's urine can smell for up to two days after consumption. Furthermore, the Parkia speciosa beans may also cause strong-smelling flatulence.

Therefore, it is the aim of the invention to provide a treatment for diabetes mellitus type 2 that is at least equally effective as the Parkia speciosa pods or beans, but which does not cause the negative side-effects described above.

This object is achieved by providing a freeze-dried fruit of Parkia obtained or obtainable by a process comprising the steps of

a) freezing a fruit from Parkia to −20° C. or below to obtain a frozen fruit and

b) subsequently freeze-drying the frozen fruit by reducing the pressure surrounding the said frozen fruit to allow water and volatiles to sublimate and/or evaporate from the frozen fruit to obtain the freeze-dried fruit of Parkia,

wherein the temperature of the fruit during freeze-drying is at most 0° C., wherein the temperature of the fruit from the beginning of step a) until the end of step b) does not exceed 20° C., more preferably does not exceed 10° C., for example does not exceed 4° C., for example does not exceed 0° C., for example does not exceed −10° C., for example does not exceed −20° C.

The freeze dried fruit from Parkia obtainable by the process of the invention can suitably be used for treatment, co-treatment and/or prevention of diseases chosen from the group of diabetes mellitus type 2, diseases related to β-amyloid plaque formation and neurodegenerative diseases, preferably of diabetes mellitus type 2, preferably diabetes mellitus type 2. The freeze-dried fruit does not have the disadvantage of the pervasive strong smell as described above. Furthermore, it was surprisingly found that this freeze-dried fruit is even more effective than raw Parkia speciosa beans in the treatment, co-treatment and/or prevention of diabetes mellitus.

Examples of diseases related to β-amyloid plaque formation and neurodegenerative diseases include but are not limited to dementia and Alzheimer's disease.

As used herein, with neurodegenerative disease is meant any type of illness that comprises failure and/or death of nerve cells in the brain.

Therefore, in a first aspect, the invention relates to a process for the preparation of a freeze-dried fruit of Parkia comprising the steps of

a) freezing a fruit from Parkia to −20° C. or below to obtain a frozen fruit and

b) subsequently freeze-drying the frozen fruit by reducing the pressure surrounding the said frozen fruit to allow water and volatiles to sublimate and/or evaporate from the frozen fruit, to obtain the freeze-dried fruit of Parkia,

wherein the temperature of the fruit during freeze-drying is at most 0° C., wherein the temperature of the fruit from the beginning of step a) until the end of step b) does not exceed 20° C., more preferably does not exceed 10° C., for example does not exceed 4° C., for example does not exceed 0° C., for example does not exceed −10° C., for example does not exceed −20° C.

In principle all fruits of plants belonging to the plant genus Parkia may be used for the preparation of the product of the invention. Examples of the Parkia plant genus include Parkia barnebyana, Parkia bahiae, Parkia bicolour, Parkia biglobosa, Parkia biglandulosa, Parkia cachimboensis, Parkia clappertoniana, Parkia decussate, Parkia discolour, Parkia filicoidea, Parkia igneiflora, Parkia inundabilis, Parkia javanica, Parkia leiophylla, Parkia lutea, Parkia madagascariensis Parkia multijuga, Parkia nana, Parkia nitida, Parkia oliveri, Parkia panurensis, Parkia paya, Parkia pendula, Parkia platycephala, Parkia reticulate, Parkia roxburghii, Parkia sherfeseei, Parkia singularis, Parkia singularis subsp. borneensis, Parkia singularis subsp. singularis, Parkia speciosa, Parkia speciosa subsp. speciosa, Parkia sumatrana, Parkia sumatrana subsp. streptocarpa, Parkia timoriana, Parkia ulei var. surinamensis, Parkia ulei var. ulei, Parkia versteeghii. Preferably, the plant is Parkia speciosa.

Preferably, the fruit is a seed-pod comprising the beans, more preferably the beans are used for the preparation of the product of the invention (the freeze-dried fruit of Parkia).

The process of the invention comprises the step of freezing a fruit from Parkia to −20° C. or below to obtain a frozen fruit.

The person skilled in the art knows how to freeze a material, for instance, the fruit may for example be directly contacted with an inert liquid gas, such as liquid nitrogen, helium or argon, preferably liquid nitrogen.

Preferably, the fruit is frozen quickly. Or in other words, the freezing method is preferably chosen such that the freezing process does not take a lot of time. To enable quick freeze-drying, the fruit may for example be cut into smaller parts before freezing. Alternatively, the fruit may be mixed with solid carbon dioxide (also known as dry ice). The mixture of dry ice and fruit may then be ground into a powder. Preferably, the temperature of the pulverized fruit is kept below −20° C. before the fruit powder is subjected to freeze-drying.

The fruit from Parkia—before subjecting it to the process of the invention—may for example be fresh, dried or frozen, preferably fresh. If frozen fruit from Parkia is used, it is preferred that the freezing of the fruit has been done quickly after harvesting, for example within 4 to 6 hours after harvesting. It is also preferred that the fruit has not been subjected to a thawing process prior to using it in the process of the invention. If fresh fruit from Parkia is used, it is preferred that the fruit is used in the process of the invention quickly after harvesting, for example within 24 hours after harvesting.

Without wishing to be bound to theory, it is believed that the active ingredient in the fruit of Parkia is not capable of withstanding elevated temperatures, which is why preferably, in the process of the invention all steps from harvesting the fruit of Parkia till in the preparation of the freeze-dried fruit of Parkia are performed at a temperature which does not exceed 30°, for example does not exceed 20° C., more preferably does not exceed 10° C., for example does not exceed 4° C., for example does not exceed 0° C., for example does not exceed −10° C., for example does not exceed −20° C.

In ‘Effects of an Indonesian medicinal plant, Curcuma xanthorrhiza Roxb., on the levels of serum glucose and triglyceride, fatty acid desaturation, and bile acid excretion in streptozotocin-induced diabetic rats’ by Yasni, S., Imaizumi, K. and Sugano, M. in Agric. Biol. Chem., 55 (12), 3005-3010, 1991, seeds of Parkia speciosa were freeze-dried, milled, and sifted through a 100-mesh sieve. However, ameliorating effects on diabetic symptoms in rats were not shown for these freeze-dried seeds. It is believed by the inventors of the present invention that the freeze-dried fruit was exposed to heat by milling the freeze-dried fruit causing the loss of pharmaceutical activity.

Preferably, the fruit used for step a) in the process of the invention has not been subjected to a temperature that exceeds 30° C., for example a temperature that exceeds 20° C. starting from the harvest of the fruit.

The freezing temperature (that is the temperature of the surroundings of the fruit) in step a) of the process of the invention is preferably at most −18° C., for example at most −20° C., for example at most −50° C., more preferably at most −80° C., for example at least −197° C. and/or for example at least −250° C.

Generally, the freezing temperature is at least −197° C. and at most −20° C.

The pressure surrounding the frozen material may be reduced using methods known to the person skilled in the art, for instance by using a vacuum pump or commercially available freeze-drying machine.

During freeze-drying the temperature of the fruit should not exceed 0° C. In order to achieve this, cooling of the fruit might be necessary. Methods known to the skilled person may be used to cool the fruit.

The process of the invention is not limited to the process steps described and may also comprise further steps or substeps, for example the thus obtained freeze-dried fruit may optionally be dried further and/or the fruit may be broken into smaller particles to facilitate further formulation into a composition, for example a pharmaceutical or nutraceutical composition.

For example, step a) of the process of the invention may further comprise the substep of breaking the frozen fruit into smaller particles and/or for example, step b) of the process of the invention may further comprise the substep of breaking the frozen fruit into smaller particles.

Further drying may for example be achieved by using a vacuum exsiccator with P₂O₅ or silica gel as a drying agent. In principle, any method may be used to break the frozen fruit or freeze-dried fruit into smaller particles, for instance the material may be grinded, crushed, pulverized, etc.

The freeze dried fruit may for example be broken into particles that have a mean diameter of less than 0.71 mm. In the context of the present invention, the ‘mean diameter’ can be determined by using a sieve having a certain mesh size; which is a method known to the skilled person.

Preferably, the obtained freeze-dried fruit (optionally further dried and/or broken into smaller particles) is stored in a dry environment to maintain pharmaceutical and/or nutraceutical activity of the freeze-dried fruit. Furthermore to maintain pharmaceutical and/or nutraceutical activity of the freeze-dried fruit, storing of the freeze-dried fruit obtained in the process of the invention until use, is preferably done at a temperature which does not exceed 30°, for example does not exceed 20° C., more preferably does not exceed 10° C., for example does not exceed 4° C., for example does not exceed 0° C., for example does not exceed −10° C., for example does not exceed −20° C. Therefore, the process of the invention may further comprise the step of storing the freeze-dried fruit of step b) until pharmaceutical or nutraceutical use at a temperature that does not exceed 30° C., for example does not exceed 20° C., more preferably does not exceed 10° C., for example does not exceed 4° C., for example does not exceed 0° C., for example does not exceed −10° C., for example does not exceed −20° C. Also, the obtained freeze-dried fruit (optionally further dried and/or broken into smaller particles) obtained in the process of the invention may be formulated into a pharmaceutical or nutraceutical composition

In another aspect, the invention relates to a freeze-dried fruit of Fabaceae, preferably Parkia obtained or obtainable by the process of the invention.

In another aspect therefore, the invention relates to a composition comprising the freeze-dried fruit of the invention. Preferably, for a single dose, the amount of freeze-dried fruit of Parkia in the composition corresponds to an amount of fresh Parkia fruit, preferably beans, in the range from 0.625 to 2.5 g.

In another aspect, the invention relates to a composition comprising freeze-dried fruit of Parkia, preferably comprising the freeze-dried product of the invention, more preferably comprising the freeze-dried product directly obtained by the process of the invention, wherein the composition is a pharmaceutical composition further comprising a pharmaceutically acceptable carrier.

A person skilled in the art knows which carriers can be used as pharmaceutically acceptable carriers. Examples of such pharmaceutically acceptable carriers are both inorganic and organic carrier materials, suitable for oral/parenteral/injectable administration and include lactose, starch, magnesium stearate, vegetable oils, and the like.

Besides freeze-dried fruit of Parkia, preferably the freeze-dried product obtainable by the process of the invention, more preferably the freeze-dried product directly obtained by the process of the invention and a pharmaceutically acceptable carrier, the pharmaceutical composition according to the present invention, may further comprise conventional pharmaceutical additives and adjuvants, excipients or diluents, including, but not limited to, water, gelatin of any origin, vegetable gums, ligninsulfonate, talc, sugars, starch, gum Arabic, vegetable oils, polyalkylene glycols, flavoring agents, preservatives, stabilizers, emulsifying agents, buffers, lubricants, colorants, wetting agents, fillers, and the like.

Preferably pharmaceutical additives, adjuvants, excipients and/or diluents are used which are dry and do not attract water (are not a hygroscopic material).

In another aspect, the invention relates to a composition comprising freeze-dried fruit of Parkia, preferably comprising the freeze-dried fruit of Parkia obtained or obtainable by the process of the invention, wherein the composition is a food or a beverage or a supplement composition for a food or beverage. Such composition is further referred to as nutraceutical composition.

Examples of fortified food include cereal bars, chewing gum and bakery items, such as cakes and cookies.

Beverages encompass non-alcoholic and alcoholic drinks as well as liquid preparation to be added to drinking water and liquid food. Non-alcoholic drinks are for instance soft drinks, sport drinks, fruit juices, such as for example orange juice and apple juice; lemonades, teas, near-water drinks and milk based drinks, such as for example yoghurt drinks. Examples of liquid food include soups and dairy products, for instance yoghurt.

The nutraceutical and pharmaceutical compositions of the present invention may be in any galenic form that is suitable for administering to the animal body including the human body, more in particular in any form that is conventional for oral administration, e.g. in solid form, for example as (additives/supplements for) food or feed, food or feed premixes, fortified food or feed, tablets, pills, granules, dragées, capsules, and effervescent formulations such as powders and tablets, or in liquid form, for instance in the form of solutions, emulsions or suspensions, for example as beverages, pastes and oily suspensions. The pastes may be filled into hard or soft shell capsules. Examples for other application forms are forms for transdermal, parenteral, topical, e.g. using patches or injectable administration. Also, the compositions may be applied using an implantable device. The nutraceutical and pharmaceutical compositions may be in the form of controlled (delayed) release formulations. Examples of pharmaceutical compositions also include compositions suitable for topical application and transdermal absorption of the phenolic compound, such as crèmes, gels, sprays, dry sticks, powders etc. Preferably, the nutraceutical or pharmaceutical compositions of the invention are in a form that is conventional for oral administration thereof as oral administration is less invasive for patients and healthy individuals than administration by injection (which is the common way to administer insulin).

A multi-vitamin and mineral supplement may be added to the nutraceutical or pharmaceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical and pharmaceutical compositions of the present invention may be used in combination with other nutraceutical compositions or therapeutic agents know to those skilled in the art for treatment, co-treatment and/or prevention of diabetes mellitus by administration prior to, simultaneously with or following the administration of the nutraceutical and pharmaceutical compositions of the present invention.

In another aspect, the invention relates to a composition comprising freeze-dried fruit of Parkia, preferably comprising the composition of the invention for use as a medicament.

In another aspect, the invention relates to use of a composition comprising freeze-dried fruit of Fabaceae, preferably comprising the composition of the invention, for the manufacture of a nutraceutical or pharmaceutical composition for the treatment, co-treatment and/or prevention of a disease diseases chosen from the group of diabetes mellitus type 2, diseases related to β-amyloid plaque formation and neurodegenerative diseases.

As used herein with ‘co-treatment’ is meant that the treatment is performed together with another pharmaceutically active ingredient.

Preferably, the invention relates to use of a composition comprising freeze-dried fruit of Fabaceae, preferably comprising the composition of the invention, for the manufacture of a pharmaceutical composition for the treatment, co-treatment and/or prevention of diabetes mellitus type 2.

In another aspect, the invention relates to a composition comprising freeze-dried fruit of Parkia, preferably comprising the pharmaceutical composition of the invention for use in the treatment, co-treatment and/or prevention of a disease chosen from the group of diabetes mellitus type 2, diseases related to β-amyloid plaque formation and neurodegenerative diseases, preferably of diabetes mellitus type 2.

In another aspect, the invention relates to a method for treatment, co-treatment and/or prevention of a disease chosen from diseases chosen from the group of diabetes mellitus type 2, diseases related to β-amyloid plaque formation and neurodegenerative diseases, preferably of diabetes mellitus type 2, preferably diabetes mellitus type 2 in animals including humans said method comprising the step of administering an effective amount of a composition comprising freeze-dried fruit of Parkia, preferably the pharmaceutical composition of the invention to animals including humans, which are in need thereof.

In the framework of the invention, with animals is meant all animals, including mammals, examples of which include humans. Preferred examples of mammals beside humans include dogs, cats, dromedaries, camels, elephants, and horses.

With ‘effective amount’ of (a composition comprising) freeze-dried fruit of Parkia, preferably the pharmaceutical composition of the invention is meant the amount needed to lower the glucose levels in the blood of the animals, including humans to which/whom the composition is administered. Preferably, the effective amount of (a composition comprising) freeze-dried fruit of Parkia also lowers the insulin requirements of the animal to which the freeze-dried fruit or (a composition comprising) freeze-dried fruit of Parkia is administered.

In another aspect, the invention relates to the use of freeze-dried fruit of Parkia, preferably of the (nutraceutical) composition of the invention, for weight regulation in animals, preferably humans, preferably wherein the use is a cosmetic use. In another aspect, the invention also relates to the use of beans of Parkia, preferably Parkia speciosa, for weight regulation in animals, preferably humans, preferably wherein the use is a cosmetic use.

Preferably the fruit is beans and/or preferably Parkia is Parkia speciosa.

The invention is now elucidated by way of the following examples, without however being limited thereto.

### EXAMPLES

### Brief Description of the Figures

FIG. 1a describes the influence of the freeze-dried product of the invention on the blood glucose levels in a female volunteer having diabetes mellitus type 2.

FIG. 1b describes the influence of the freeze-dried product of the invention on the insulin requirements of a female volunteer having diabetes mellitus type 2.

FIG. 2a describes the influence of residue of the Parkia speciosa beans after extraction with n-hexane on the blood glucose levels in a female volunteer having diabetes mellitus type 2.

FIG. 2b describes the influence of residue of the Parkia speciosa beans after extraction with n-hexane on the insulin requirements of a female volunteer having diabetes mellitus type 2.

FIG. 3a describes the influence of extract that was collected from the n-hexane fraction used for extraction of Parkia speciosa beans on the blood glucose levels in a female volunteer having diabetes mellitus type 2.

FIG. 3b describes the influence of the freeze-dried product of extract that was collected from the n-hexane fraction used for extraction of Parkia speciosa beans on the insulin requirements of a female volunteer having diabetes mellitus type 2.

FIG. 4. shows the blood glucose levels of C57BI6 mice that received intragastrically 0, 4 or 13 mg/kg bodyweight of extract of example A.

FIG. 5 shows the blood insulin levels of C57BI6 mice that received intragastrically 0, 4 or 13 mg/kg bodyweight of extract of example A.

### Experiment 0

Prior to experiments 1-3 with capsulated products from Parkia speciosa beans (as described below) a female volunteer having diabetes mellitus type 2 took fresh Parkia speciosa beans, ¼ bean at a time, just before taking three regular daily meals. During a three months period blood glucose levels of the female volunteer were perfectly under control and no adverse side-effects were observed apart from the pervasive smell that is associated with the Parkia speciosa beans. This experiment confirmed that fresh beans of Parkia speciosa are suitable for treatment of diabetes mellitus type 2.

Corresponding amounts: all medication was administered such that the dose of freeze-dried beans given was in correspondence with the dose of raw beans used in experiment 0. In other words, the amount of material in a single dose freeze-dried beans was similar to the amount of material in a single dose of raw beans (¼ raw/fresh beans per dose).

### Example A

### Freeze-dried Product of Parkia speciosa

The weight of 15 mean-sized fresh beans was determined 38 grams. Thus, 25.5 g corresponds with 15×25.5/38=10 beans. For application of intact unprocessed beans, a single dose concerns ¼ bean.

Freeze-drying of 50 gram fresh beans, which corresponds with 20 beans (calculated by 15×50/38) yielded 8.90 gram freeze-dried bean material, which are 80 doses, a single dose being 111 mg.

For oral administration, 40 capsules No 1 were prepared, which have a total volume of 18.5 ml. A 25 ml-measuring cylinder was put on a balance that was subsequently adjusted to zero. An amount of 4.45 gram freeze-dried (in a commercially available freeze-drying machine at a temperature below 0° C.) and pulverized bean material (pulverization was done by grinding in a mortar at room temperature; about 20° C.) transferred into the cylinder, showed a volume of 8.5 ml. Microcrystalline cellulose (according the European Pharmacopoeia) was then added in portions with occasional tapping at the cylinder up to 17.5 ml. Hereafter, the cylinder's contents were very well mixed by use of mortar and pestle. The mixture obtained was transferred in portions to the cylinder, and microcrystalline cellulose was added up to the final volume of 18.5 ml. Mixture and cellulose were added in portions with tapping at the cylinder in between for compression of the materials. Weighing the cylinder with its contents showed an amount of 4.55 gram microcrystalline cellulose totally added. All cylinder's contents were transferred to the mortar and well mixed before all material was divided over 40 capsules using a capsule-filling device. Each capsule contained a dose of 111 mg freeze-dried bean material, corresponding with ¼ bean.

### Example B

### Extraction with n-hexane; Residue

Using a Ultra-turrax machine, 25.5 g of fresh beans were mashed and intensively mixed with analytical-grade n-hexane for 5 minutes and room temperature (20-25° C.). The hexane extract that is n-hexane solution containing extracted lipophylic bean constituents, was decanted. Subsequently, fresh hexane was added to the mash and above-described procedure was repeated four times. So, for removal of smelling constituents, mashed fresh beans were extracted five times with n-hexane. In other words, fresh beans were exhaustively extracted using n-hexane as solvent for a polar substances. At the end of each extraction, most of the residual hexane was removed by pressing the mash together using a spatula.

The hexane extracts were combined, and the solvent removed under reduced pressure using a rotary evaporation apparatus. An oily residue was obtained, yellowish-green that stinked quite disgustingly, indicating that most if not all undesired, smelly components had been removed from the beans.

The mashed and extracted beans that were almost free of hexane were dried in an oven at a temperature above 35° C., but not exceding 39° C. After 3 hours, the dry product was kept overnight in a desiccator above concentrated sulfuric acid, before further processing.

Dried beans were now pulverized at room temperature (20-25° C.) using mortar and pestle. All ground material was put through a seive (mesh size 0.71 mm). In other words, the powder of dry extracted beans concerned material with a particle size≦0.71 mm.

The powdered material was kept for 24 hours in a desiccator above concentrated sulfuric acid. Finally, 3.19 g dry powder was obtained out of 25.5 g of fresh beans.

The weight of 15 mean-sized fresh beans was determined 38 grams. Thus, 25.5 g corresponds with 15×25.5/38=10 beans. For application of intact unprocessed beans, a single dose concerns ¼ bean.

So, 3.19 g dry powder that was obtained from 25.5 gram fresh beans, represents 10 fresh beans, which are 40 doses by ¼ bean being a single dose. For beans processed by hexane extraction and subsequent drying and pulverizing, a single dose was calculated by 3.19/40, i.e. 0.08 g.

For oral administration, capsules were prepared, using capsules No 1. The volume of 40 capsules No.1 is 18.5 ml. A 25 ml-measuring cylinder was put on a balance that was subsequently adjusted to zero. Using the cylinder, 3.19 gram processed beans showed a volume of 6 ml. Microcrystalline cellulose (according the European Pharmacopoeia) was then added in portions with occasional tapping at the cylinder up to 17.5 ml. Hereafter, the cylinder's contents were mixed lege artis using mortar and pestle. The mixture obtained was transferred in portions to the cylinder, and microcrystalline cellulose was added up to the final volume of 18.5 ml. Mixture and cellulose were added in portions with tapping at the cylinder in between for compression of the materials. Weighing the cylinder with its contents showed an amount of 6.3 gram microcrystalline cellulose totally added. All cylinder's contents were transferred to the mortar and well mixed before all material was divided over 40 capsules using a capsule-filling device.

### Example C

### Extraction with n-hexane; Hexane Fraction

Parkia speciosa beans were freeze-dried as described above. This, for removal of undesired smelly constituents. The freeze-dried beans were ground into a powder a particle size≦0.71 mm. An amount of 2.00 gram was transferred into a screw-cap vial and 7.5 ml of n-hexane was added. The closed vial was stored in a freezer at −20° C. for 24 hours to allow extraction of lipophylic constituents from the bean material.

Then, n-hexane containing lipophylic components or, in other words, hexane extract, was decanted and filtered through a paper filter. The residue (that is extracted freeze-dried and pulverized Parkia beans) was washed three times with 5 ml of n-hexane. Using the same filter, the hexane washings were filtered, as well. The decanted and filtrated extract and washings were collected in a round-bottom flask of known weight; n-hexane was removed under reduced pressure.

A small amount of residue left behind on the paper filter after the last washing was returned into the screw-cap vial. After addition of 7.5 ml of n-hexane, the closed vial was stored in the freezer for a second 24 hour-period of extraction. Decantation and washings were performed as above. The second filtrated extract and corresponding washings were collected in the same round-bottom flask with residues of the first extract and washings already present. After evaporation of hexane under reduced pressure, weighing showed the flask to contain 0.50 gram extractives.

The above-described procedure resulted in an extract containing lipophylic constituents of Parkia speciosa beans but without any smelling components present. After taking 20 mg as reference material, 0.48 gram n-hexane extract was available for processing into capsules.

As freeze-drying of 50 gram fresh beans yielded 8.90 gram freeze-dried material, which corresponds with 20 beans, ¼ bean being a single dose, 0.48 gram n-hexane extract obtained from 2.0 gram dry beans represented 17 single doses. This was calculated by (20×2.00/8.90)×4×(0.48/0.50).

For capsulation, 17 capsules No. 1 were used having a total content of 8.5 ml. Diethylether was added to the round-bottom flask to dissolve the hexane extract (0.48 gram). The solution was quantitatively transferred to a mortar that was covered during the washing steps to limit evaporation of diethyl ether. Then 3.0 gram of microcrystalline cellulose was added to the solution. Immediately after, thorough mixing was applied until all diethylether had evaporated and mortar and pestle were used at room temperature.

A 10 ml-measuring cylinder was put on a balance that was subsequently adjusted to zero. Then, the mixture of cellulose (3.0 gram) and hexane extract (0.48 gram) was transferred to the cylinder, followed by microcrystalline cellulose up to 8.5 ml. Both were added in portions with tapping at the cylinder in between for sufficient compression of the contents. Weighing showed a content of 3.7 gram. So, the total amount of microcrystalline cellulose added was 3.22 gram (3.7-0.48).

All cylinder's contents were transferred to the mortar and well mixed before all material was divided over 17 capsules using a capsule-filling device. Each capsule contained 28 mg hexane extract corresponding with a single dose of ¼ bean. The hexane extract lacked the pervasive smell associated with fresh beans.

### Administration of Capsules to Female Human Volunteer with Diabetes Mellitus Type 2

### Experiment 1

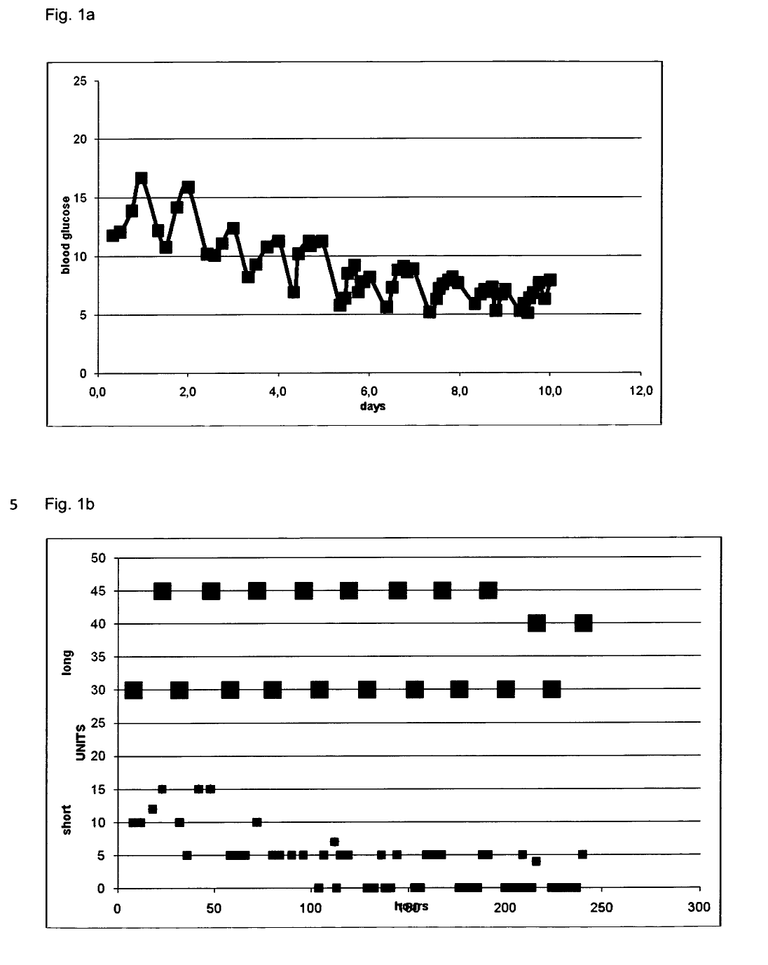

The same female volunteer of experiment 0 ingested (not simultaneously with any other of the experiments described herein) the capsules of example A, three times per day prior to three regular meals. Blood glucose levels were measured. Insulin intake, based on the measured glucose level was written down. The results are shown in FIGS. 1a and 1b (FIG. 1a, FIG. 1b).

### Experiment 2

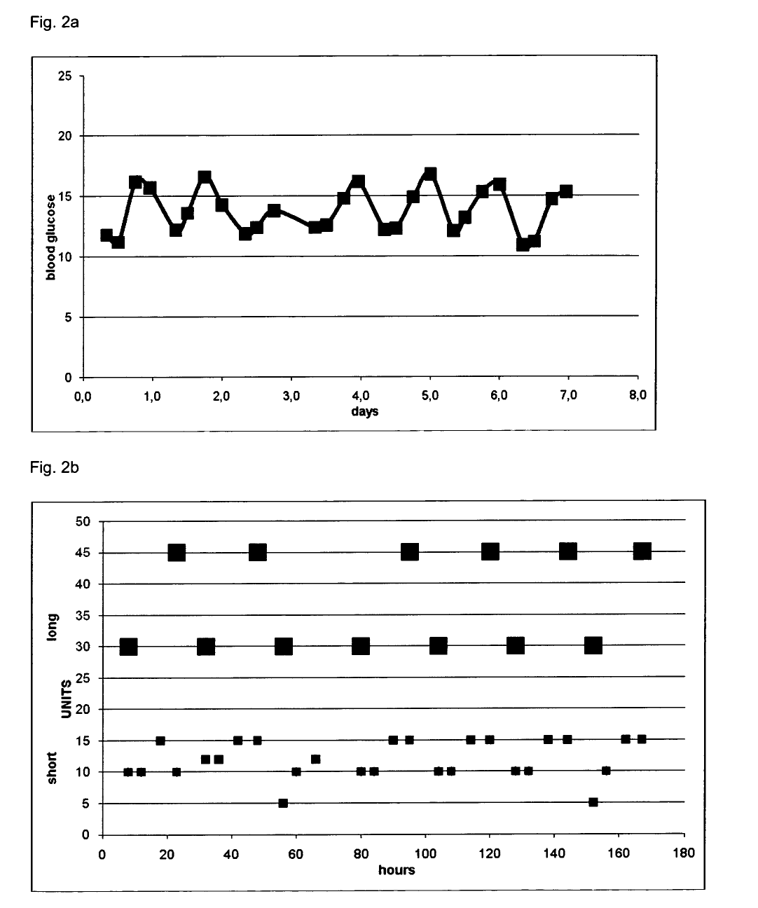

The same female volunteer of experiment 0 ingested (not simultaneously with any other of the experiments described herein) the capsules of example B above, three times per day prior to three regular meals. Blood glucose levels were measured. Insulin intake, based on the measured glucose level was written down (hereafter also indicated as ‘insulin requirements’). The results are shown in FIGS. 2a and 2b (FIG. 2a, FIG. 2b).

### Experiment 3

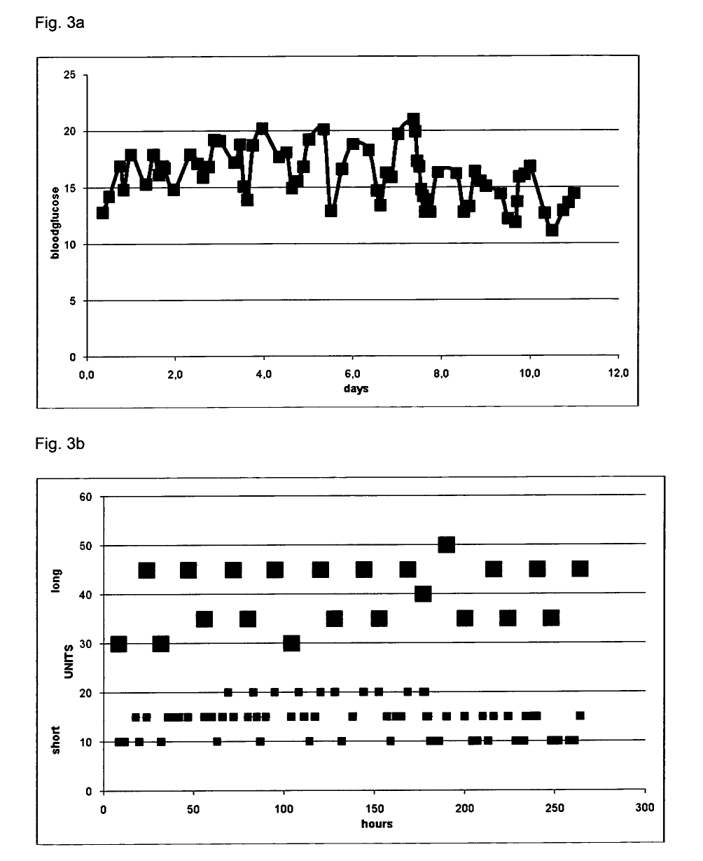

The same female volunteer of experiment 0 ingested (not simultaneously with any other of the experiments described herein) the capsules of example C above, three times per day prior to three regular meals. Blood glucose levels were measured. Insulin intake, based on the measured glucose level was written down. The results are shown in FIGS. 3a and 3b (FIG. 3a, FIG. 3b).

### Discussion and Conclusion of Experiments 1-3

As can be seen from FIG. 1a within two days after intake of freeze-dried beans of Parkia speciosa, the blood glucose levels of the female volunteer decreased. Also, as can be seen from FIG. 1b, the insulin requirements decreased. When intake of the fresh beans was stopped (and no other medication was taken besides insulin), both glucose levels and insulin requirements increased to the normal levels of the female volunteer. Therefore, experiment 1 confirmed that freeze-dried beans of Parkia speciosa according to the invention are suitable for treatment of diabetes mellitus type 2.

Also, no side-effects were observed and surprisingly, the pervasive smell that was present when using the fresh beans in experiment 0, was absent in experiment 1. Furthermore, it was also surprisingly observed that the product of the invention has an even bigger impact on lowering the glucose levels and insulin requirements than the fresh beans of Parkia speciosa of experiment 0, even though the equivalent amount ingested by the female volunteer was the same.

After stopping the intake of the product of the invention (and by taking no other medication besides insulin) of which experiment 1 is a non-limiting example, both glucose levels and insulin requirements increased to the levels normal for the female volunteer.

Therefore, it can be concluded that the freeze-dried product of the invention is suitable for use in the treatment of diabetes mellitus type 2. Surprisingly this product does not have the smell and was proven to be more effective than the treatment using the fresh beans of Parkia speciosa.

As can be seen from FIG. 2a and FIG. 2b, intake of the residue of the beans after extraction with n-hexane had the effect that glucose levels and insulin requirements remained the same. Hence, the residue of the beans after extraction with n-hexane is not suitable for treatment of diabetes mellitus type 2, even though the smell was also not observed in experiment 2.

As can be seen from FIG. 3a and FIG. 3b, intake of the extract that was collected from the n-hexane fraction used for extraction of Parkia speciosa beans, has the effect that glucose levels and insulin requirements remained the same. Hence, the extract that was collected from the n-hexane fraction is not suitable for treatment of diabetes mellitus type 2, even though the smell was also not observed in experiment 3.

The effect of the product of the invention, as exemplified by the product of example A, on the glucose levels and insulin requirements was also tested in mice.

### Experiment 4

### Influence of Freeze-dried Parkia speciosa Beans/seeds (Example A) on the Blood Glucose Levels of Mice

Normal 7 weeks old C57BI6 male mice (n=13) were put on a reversed day/night schedule one week before start of the experimental fase to acclimatize. For fourteen days, two times a day at 08:00 hours and at 14:00 hours extract was given intragastrically (100 μl each time). 5 mice received two times a day 4 mg extract/kg bodyweight, 5 mice received two times a day 13 mg extract/kg bodyweight and 3 mice (the control group) received two times a day carboxy methyl cellulose in water only. Approximately 75 μl of blood was collected via cheek sting at day 1 prior to the first extract dosage, at day 7 prior to the 14:00 hours dosage and at day 14, prior to the 14:00 hours dosage. Blood was collected in EDTA (anticoagulant) containing tubes; the tubes were spun in a centrifuge, subsequently, the plasma was taken with a pipette and the samples were stored at −80° C. until glucose and insulin levels could be determined. At day 16 all animals were anesthetized with carbon dioxide, a heart puncture was performed for blood collection and finally the animals were killed by cervical dislocation. Pancreas, kidneys, liver and spleen were taken out, partly fixated in formalin, and partly deeply frozen immediately in liquid nitrogen and stored at −80° C. The health status in general and the above mentioned organs were studied and judged by a pathology specialized veterinarian.

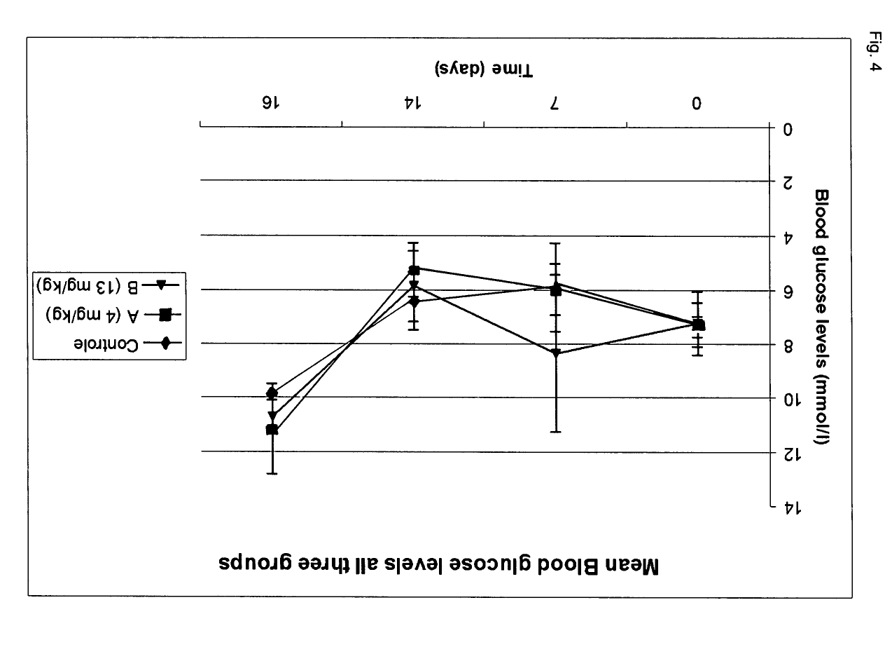

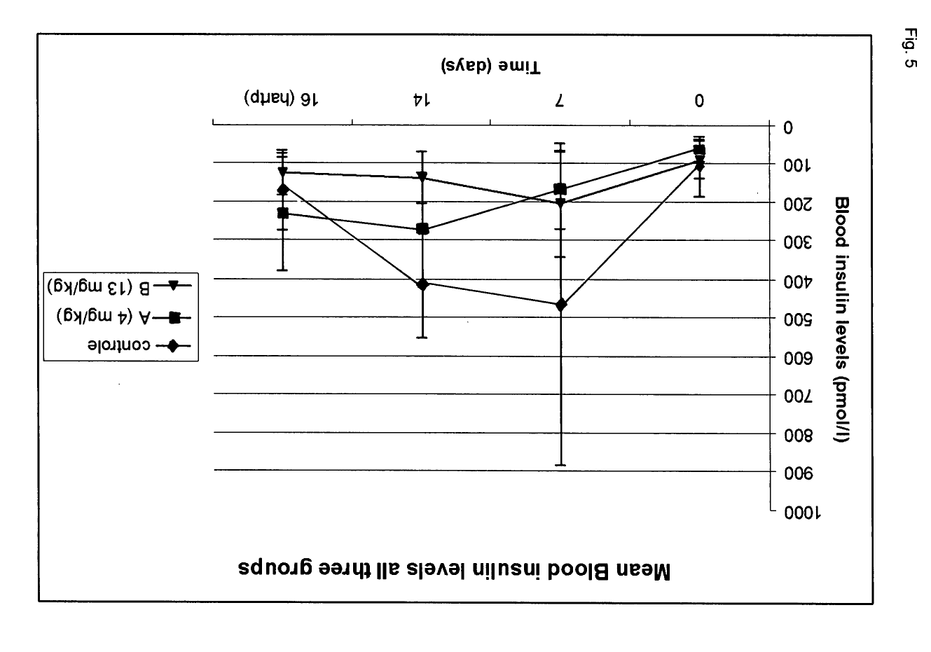

The results of this experiment are shown in FIGS. 4 and 5.

### Results, Discussion and Conclusion of Experiment 4

Autopsy revealed that the general health status of animals was perfectly in order and did not show any abnormalities. Therefore it can be concluded that the extract, which was given either 2×4 mg/kg or 2×13 mg/kg per day for 14 days is not toxic to mice.

As can be seen from FIG. 4, after 14 days the plasma glucose levels of the mice that were administered 4 mg/kg respectively 13 mg/kg of the product of example 1 as compared to the control group did not significantly decrease the plasma glucose levels of the mice. In other words, dosage of the extract did not have a significant effect on the blood glucose level of the mice that were administered the extract as compared to the control mice.

As can be seen from FIG. 5, the mice that were administered 4 mg/kg respectively 13 mg/kg of the product of example 1 as compared to the control group had a significantly lower plasma insulin level. In the 13 mg/kg group the plasma insulin levels were a lot lower at day 14 (p=0.007). Since their glucose plasma levels remain the same (FIG. 4), this indicates that the product of the invention sensitizes the mice for insulin, thereby having the effect that the mice need less insulin to control their blood glucose levels.

Therefore, experiment 4 indicates that the product of the invention is suitable for the prevention, treatment and/or co-treatment of diabetes mellitus, in particular of diabetes mellitus type 2.

### Effect of Fruit of Parkia on Weight Regulation

Furthermore, the female volunteer observed that during the period that she took fresh Parkia speciosa beans prior to her meal, she had a lack of apetite resulting in weight loss. Also, in experiment 4 it was observed that the mice did not gain any weight during the 14 day treatment, whereas normally 2 to 3 g of weight gain was expected. Therefore it is concluded that the fruit, preferably beans of Parkia, preferably Parkia speciosa beans and the freeze-dried product of the invention could play a role in weight regulation.

1-19. (canceled)

20. A process for the preparation of a freeze-dried fruit of Parkia, comprising:
a) freezing a fruit from Parkia to −20° C. or below to obtain a frozen fruit, and
b) subsequently freeze-drying the frozen fruit by reducing the pressure surrounding the frozen fruit to allow water and volatiles to sublimate and/or evaporate from the frozen fruit to obtain the freeze-dried fruit of Parkia,
wherein the temperature of the fruit during freeze-drying is at most 0° C., and
wherein the temperature of the fruit from the beginning of step a) until the end of step b) does not exceed 30° C.

21. The process of claim 20, wherein the temperature of the fruit from the beginning of step a) until the end of step b) does not exceed 25° C.

22. The process of claim 21, wherein the temperature of the fruit from the beginning of step a) until the end of step b) does not exceed 10° C.

23. The process of claim 22, wherein the temperature of the fruit from the beginning of step a) until the end of step b) does not exceed 4° C.

24. The process of claim 23, wherein the temperature of the fruit from the beginning of step a) until the end of step b) does not exceed 0° C.

25. The process of claim 20, wherein the fruit is cut into smaller pieces prior to freezing.

26. The process of claim 20, further comprising breaking the frozen fruit into smaller particles.

27. The process of claim 20, further comprising breaking the freeze-dried fruit into smaller particles.

28. The process of claim 20, wherein the fruit has not been subjected to a temperature that exceeds 30° C. starting from the harvest of the fruit.

29. The process of claim 20, further comprising storing the freeze-dried fruit at a temperature that does not exceed 30° C. until pharmaceutical or nutraceutical use.

30. The process of claim 20, wherein the Parkia is Parkia speciosa, and/or wherein the freeze-dried fruit is a bean.

31. A freeze-dried fruit of Parkia, obtainable by the process of claim 20.

32. A composition comprising the freeze-dried fruit of claim 31.

33. The composition according to claim 32, wherein, for a single dose, the amount of freeze-dried fruit corresponds to an amount of fresh Parkia fruit in the range of from 0.625 g to 2.5 g.

34. The composition according to claim 32, wherein the composition is a pharmaceutical composition further comprising a pharmaceutically acceptable carrier.

35. A method for the treatment, co-treatment and/or prevention of a disease selected from the group consisting of diabetes mellitus type 2, diseases related to β-amyloid plaque formation and neurodegenerative diseases, the method comprising administering to a mammal in need thereof an effective amount of a freeze-dried fruit of Parkia according to claim 31.

36. The method of claim 35, wherein the disease is diabetes mellitus type 2.

37. The method of claim 35, wherein the mammal is a human.

38. A method for regulating the weight of a mammal, comprising administering to a mammal in need thereof an effective amount of a freeze-dried fruit of Parkia according to claim 31.

39. The method of claim 38, wherein the mammal is a human.
